(19) United States
(12) Patent Application Publication
(10) Pub. No.: US 2006/0142683 A1
(43) Pub. Date: Jun. 29, 2006

(54) Spinal decompression therapy system and method

(75) Inventors: Scot Johnson, Lutz, FL (US); James J. Gibson JR., Tampa, FL (US); Nicholas James Exharos, Tampa, FL (US)

Correspondence Address: MCANDREWS HELD & MALLOY, LTD, 500 WEST MADISON STREET, SUITE 3400, CHICAGO, IL, 60661, US

(73) Assignee: Axiom Worldwide, Inc.

(21) Appl. No.: 11/054,814

(22) Filed: Feb. 10, 2005

Related U.S. Application Data
(60) Provisional application No. 60/639,916, filed on Dec. 28, 2004.

(51) Int. Cl. A61F 5/00

(52) U.S. Cl. 602/32; 602/36

(57) ABSTRACT

The present invention provides a system and method of modifying a treatment profile in spinal decompression therapy. The method includes applying a plurality of cycles of the treatment profile and altering at least one parameter of the treatment profile in at least one of the cycles. The parameters of a treatment profile include a high tension level, a low tension level, a high tension time period, and a low tension time period. Each of the cycles includes an application of a tensile force at a high tension level for a high tension time period and an application of the tensile force at a low tension level for a low tension time period.

### BACKGROUND OF THE INVENTION

The present invention generally relates to systems and methods for implementing spinal decompression therapies. Specifically, the present invention relates to an improved system and method for decreasing paraspinal muscle opposition to tensile forces in spinal decompression therapy.

Spinal decompression therapy is utilized to treat various spinal ailments including herniated discs, degenerative disc disease, sciatica, posterior facet syndrome, and post surgical pain, for example. Decompression therapy is a derivative of traction-based therapy, which includes placing a patient's spine in a state of tension. The state of tension is created by an outside force such as a therapist manually applying or by an automated process applying tension to the patient's spine. In traction-based therapy, the spine is held in a continuous state of tension. Decompression therapy differs from traction-based therapy in that the traction applied to the spine in decompression therapy is typically alternated between lower and higher levels of tension for predetermined periods of time. In either therapy, spinal tension is maintained for periods typically extending 30-minutes or longer.

As the spine is placed into a state of tension, the spinal vertebras are separated allowing the intervertebral discs to realign into their proper positions. This action also allows herniated discs time to heal in a non-loaded state. In addition, nutrient-rich spinal (nucleus pulposa) fluid is drawn to the sites of tension due to the pressure drop created by the tensile forces.

However, paraspinal muscle contractions may oppose the tensile force applied during decompression therapy. For example, paraspinal muscles can react involuntarily to the “stretching” of the spine by tensing in opposition to the force, otherwise known as paraspinal muscle opposition. The patient may also voluntarily flex the spinal muscles in reaction to the tensile forces. Either or both scenarios degrade the effectiveness of spinal therapy.

Current systems and methods may partially decrease the amount of paraspinal opposition forces. For example, current decompression therapy, as opposed to earlier forms of traction-based therapy, can reduce the amount of paraspinal muscle opposition by cycling the tensile forces throughout a spinal decompression treatment period. Such a cyclic application of tensile forces can confuse the paraspinal muscles, thereby decreasing the amount of paraspinal muscle opposition.

In general, paraspinal muscles are more opposed to abrupt changes in tensile forces than to gradual changes. Therefore, current decompression therapies may employ any of the family of non-linear, curvilinear, sinusoidal, exponential, or logarithmic mathematical functions in the transition between different levels of tension. In other words, current decompression therapies may use any of a non-linear, curvilinear, sinusoidal, exponential, or logarithmic mathematical function to describe the transition between low and high tensile levels versus time. By doing so, the transition between the different levels of tensile forces becomes smoother, and the amount of paraspinal muscle opposition forces is slightly decreased.

Conventional decompression systems and methods incorporate both the concepts of cycled tension (or the cyclic change between low and high levels of tension) and gradual tension transitions, as described above. However, the effect of these systems and methods on paraspinal muscle opposition is capable of being extended to a much greater extent. In other words, while current spinal decompression systems and methods do decrease paraspinal muscle opposition forces, these systems and methods are limited in the amount of paraspinal muscle opposition that they may decrease. Therefore, a need exists for a system and method for further decreasing paraspinal muscle opposition forces in spinal decompression therapy.

### BRIEF SUMMARY OF THE INVENTION

The present invention provides a method of modifying a treatment profile in spinal decompression therapy. The method includes applying a plurality of cycles of the treatment profile and altering at least one parameter of the treatment profile in at least one of the cycles. The parameters of a treatment profile include a high tension level, a low tension level, a high tension time period, and a low tension time period. Each of the cycles includes an application of a tensile force at a high tension level for a high tension time period and an application of the tensile force at a low tension level for a low tension time period.

The present invention also provides a computer-readable storage medium including a set of instructions for a computer. The instructions include a spinal decompression treatment profile generating routine and a treatment cycle adjustment routine. The generating routine is configured to calculate a plurality of cycles of a spinal decompression treatment profile. Each of the cycles includes an application of a tensile force to a spine at a high tension level for a high tension time period and an application of the tensile force to the spine at a low tension level for a low tension time period. The adjustment routine is configured to alter at least one of the high tension level, high tension time period, low tension level, and low tension time period in at least one of the cycles.

The present invention also provides a spinal decompression system for applying a treatment profile in spinal decompression therapy. The system includes a patient interface device and a control system. The interface device is capable of being employed to apply a tensile force to a spine throughout a plurality of cycles in the treatment profile. Each of the cycles includes an application of the tensile force at a high tension level for a high tension time period and an application of the tensile force at a low tension level for a low tension time period. The control system is configured to adjust the tensile force applied by the patient interface device by altering at least one of the high tension level, high tension time period, low tension level, and low tension time period in at least one of the cycles.

### BRIEF DESCRIPTION OF SEVERAL VIEWS OF THE DRAWINGS

FIG. 1 illustrates an exemplary spinal decompression system according to an embodiment of the present invention.

FIG. 2 illustrates an exemplary treatment profile for spinal decompression therapy in accordance with an embodiment of the present invention.

FIG. 3 includes a flowchart for a method of implementing a spinal decompression profile in accordance with an embodiment of the present invention.

FIG. 4 illustrates an exemplary spinal decompression treatment profile that includes the modification of a gradual treatment profile in accordance with an embodiment of the present invention.

FIG. 5 illustrates an exemplary spinal decompression treatment profile that includes the modification of tension amplitude modulation in accordance with an embodiment of the present invention.

FIG. 6 illustrates an exemplary cycle of spinal decompression treatment profile with the further modification of varying an amount of tension amplitude modulation in accordance with an embodiment of the present invention.

FIG. 7 illustrates an exemplary spinal decompression treatment profile that includes the modification of frequency modulation of high and low tensile time periods in accordance with an embodiment of the present invention.

FIG. 8 illustrates an exemplary cycle of spinal decompression treatment profile that includes the modification of frequency modulation of tension amplitude modulation in accordance with an embodiment of the present invention.

FIG. 9 illustrates an exemplary spinal decompression treatment profile that includes the modification of dynamic smoothing in accordance with an embodiment of the present invention.

FIG. 10 illustrates a treatment profile applying a corrective tensile force without dynamic smoothing in accordance with an embodiment of the present invention.

FIG. 11 illustrates a method for adjusting a treatment profile in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

Modifications of the conventional protocols utilized by commercially available spinal decompression technology described in this application are directed towards further decreasing paraspinal muscle opposition to the tensile forces applied to the spine during decompression therapy. Any of the modifications can be used alone or in any combination together. All modifications can be represented by and embodied in adjustment parameters. The adjustment parameters can be predetermined and/or updated or calculated in real-time by the system.

One modification to a conventional spinal decompression therapy is the application of a Gradual Treatment Profile. In general, a Gradual Treatment Profile includes a plurality of cycles of gradually increasing low and high tensile forces applied to a patient's spine. Each cycle of the Gradual Treatment Profile includes a low tensile level and a high tensile level. Each level represents an amount of tensile force applied to a spine. As described in more detail below, the tensile force applied to a spine is set at the low tensile level for a low tensile level period of time and then increases over a transition time period to the high tensile level. The tensile force is applied to the spine at the high tensile level for a high tensile time period, and then returns to the low tensile level for the next cycle in the Gradual Treatment Profile.

The initial low and high tensile levels (in other words, the tensile levels for the first cycle of the treatment profile) begin at an initial lowest setting, or level, for each. As the number of cycles increases, the low and high tensile levels increase until the low and high tensile levels each reach a respective predetermined maximum, thereby resulting in a gradual increase in low and high tensile forces applied to a spine. The gradual increases in low and high tension levels reduces the paraspinal muscles' involuntary opposition to abrupt changes in tension and allows a psychological adjustment period to the therapy, thereby decreasing voluntary spinal muscle opposition.

The initial low and high tension levels, or plateaus, may be input by a user or calculated as a part or percentage of a predetermined maximum low and high tension levels, or plateaus. From the initial, lowest levels of the Gradual Treatment Profile, the low and high tension levels (and thus the actual tensile force applied to the spine) are increased as the number of treatment cycles increases. After some predetermined number of cycles, the system may achieve the predetermined maximum low and high tension levels. The system may then continue to apply additional treatment cycles of alternating low and high tensile forces at these predetermined maximum levels.

Another modification to a conventional spinal decompression therapy is the application of an amplitude- and/or frequency-modulated tension level. Amplitude- and/or frequency-modulated tension levels can confuse and relax the involuntary opposition of paraspinal muscles to decompression therapy. In addition, such modulated tension levels may also create a more natural, less exact or machine-like feel to the therapy.

Amplitude modulation is a process whereby the amplitude of tension level or the tension applied to a spine is alternated by any of the family of non-linear, curvilinear, sinusoidal, exponential, or logarithmic mathematical functions, for example. In general, amplitude is a measure of the tension applied to a spine in decompression therapy. The amplitude may be varied with respect to time. The rate of alternation of the amplitude with respect to time is the frequency of the amplitude modulation.

The amplitude modulation may be subtracted from predetermined low and high tension plateau levels (described above). In doing so, a user of the system may be ensured that the tension applied to a patient's spine does not exceed the predetermined low and high tension levels.

The amount, or range, of amplitude modulation can be varied by any of the family of non-linear, curvilinear, sinusoidal, exponential, or logarithmic mathematical functions, for example.

Frequency modulation can be applied to one or more of a length of time spent at the low and high tension plateau level cycles (or low tension time period and high tension time period, respectively) or to the frequency of the amplitude modulation. Frequency modulation can exacerbate the effect of paraspinal muscle confusion by increasing the non-repeating or non-constant nature of the tension forces.

Amplitude and frequency modulation rates may be independently adjusted and controlled. One or more of amplitude and frequency modulation may apply to tension force plateaus (as described above) as well as the tension forces applied to a spine during the transition between low and high tension levels (and vice-versa).

Another modification to a conventional spinal decompression therapy is the application of dynamic smoothing to changes in tension forces during therapy. Dynamic smoothing of one or more changes in tension forces during decompression therapy can relax the involuntary opposition of paraspinal muscles' to decompression therapy and may create a more natural, less exact or machine-like feel to the therapy.

Dynamic smoothing can apply to any one or more of expected or predetermined changes in tension forces and to unexpected and likely measured changes in tension forces. Unexpected changes in tension forces are a common occurrence during decompression therapy. These can be a result of patient body shifting or external forces acting on patient or on patient interface device (for example, a tension strap connecting an electro-mechanical actuator to a patient harness setup).

The changes in tension forces may be measured by a feedback system. For example, a tensile force is expected to be applied to a patient's spine. However, the tensile force actually applied to the patient's spine may differ from the expected tensile force. A feedback system may determine the difference between the expected and actual tensile forces and communicate this difference to a control system. The control system may then adjust the electro-mechanical actuator output in order to correct the actual tensile force applied to a patient's spine, for example. After the actual tensile force is corrected, the corrected actual force may more closely match the expected tensile force.

The control system can tailor the correcting tensile force issued by the electro-mechanical actuator such that the correction to the actual tensile force is smooth and non-abrupt. The dynamic smoothing of the actual tensile forces may be calculated and applied in real-time during the therapy. In addition, any part or whole of a tensile force change (or any combination thereot) may be dynamically smoothed.

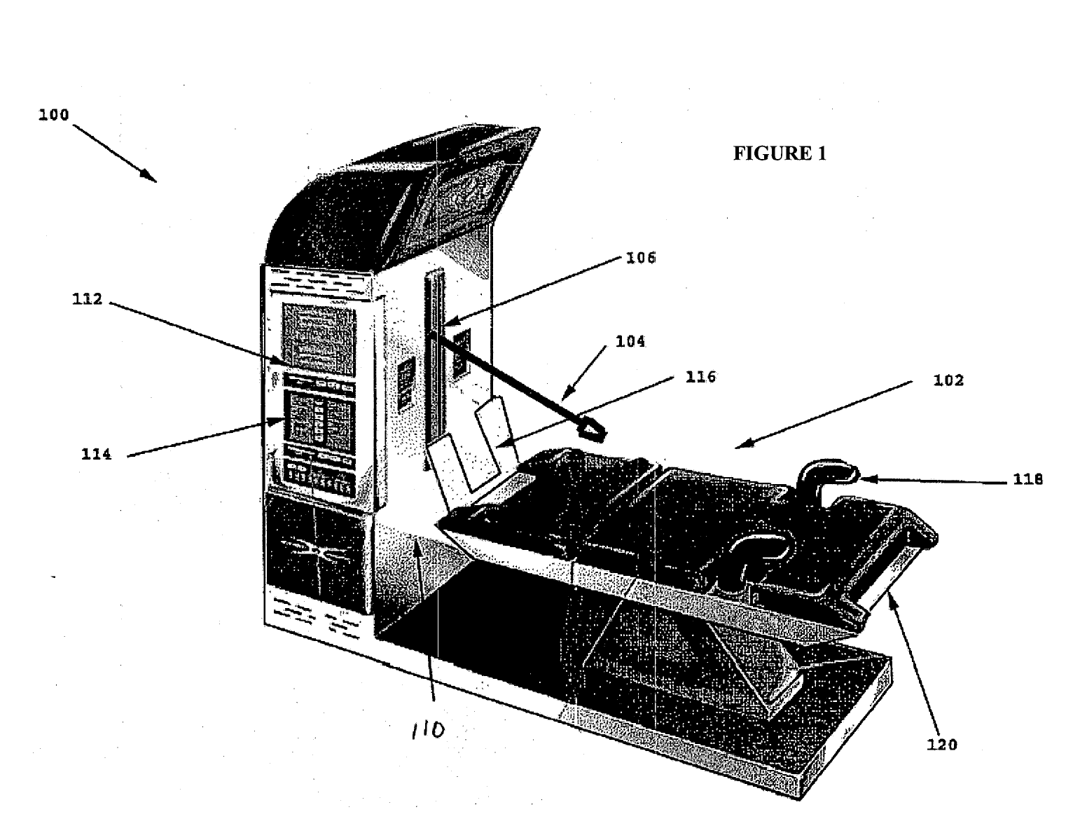

Any one or more of the above modifications to conventional therapy protocols may be implemented by an improved spinal decompression system. FIG. 1 illustrates an exemplary spinal decompression system 100 according to an embodiment of the present invention. System 100 includes a bed or table 102, a patient interface device 104, a patient interface positioning device 106, a feedback system 108 (not shown), an actuator 110, a patient interface system 112, and a control system 114.

Bed 102 includes any surface capable of supporting a patient during spinal decompression therapy. Bed or table 102 includes a head end 120 and a tail end 116. Bed 102 may be positioned so a patient can be placed into alignment for spinal decompression treatment. Bed or table 102 may include arm supports 118 or rails to position the patient.

Patient interface device 104 includes any connecting device capable of transferring a tensile force from actuator 110 to a patient harness device in order to apply the tensile force to the patient's spine. For example, patient interface device 104 can include a rod capable of being attached or connected to a patient harness device. The interface 104 and harness can deliver and align tensile forces generated by actuator 110. Interface device 104 may itself be moved to preferred positions by additional actuators (not shown).

Patient interface positioning device 106 includes any device capable of acting as a moving fulcrum for interface device 104. As illustrated in FIG. 1, patient interface positioning device 106 includes a high strength strap that is connected to a patient and to the actuator 110. Patient interface device 104 is configured to deliver or capable of delivering tensile force(s) to the patient from actuator 110 over a fulcrum (for example, a smooth axle) located within patient interface positioning device 106. Patient interface positioning device 106 can be capable of moving vertically up or down, having its lowest point nearer to the surface of table 102 and its highest point nearer to the uppermost portions of device 106, for example. By changing the position of patient interface positioning device 106, the angle from which tensile force is delivered to the patient can be adjusted with respect to the patient. Changing the angle from which tensile force is delivered to the patient may change the location of the decompression therapy in the lumbar spine, for example.

Actuator 110 includes any mechanical device configured to transfer a tensile force to a patient's spine through patient interface device 104. For example, actuator 110 can include any electro, hydraulic, pneumatic, or mechanical actuator connected to patient interface device 104. Actuator 110 may be controlled by control system 114 through a system of gears and/or pulleys so that tensile forces generated by actuator 110 and transferred to the patient's spine via patient interface device 106 would be tightly controlled.

Control system 114 includes any computing device capable of running software that performs any one or more of the previously described modifications to traditional spinal decompression therapies. For example, control system 114 can include a microprocessor or other computing device with firmware and/or software that is configured to control actuator 110. For example, control system 114 may include a computer-readable storage medium that includes one or more sets of instructions for a computing device. The set(s) of instructions includes a spinal decompression treatment profile generating routine, a treatment cycle adjustment routine, and a tensile force feedback routine. One or more of the generating routine, adjustment routine, and feedback routine may be embodied in one or more software applications.

The generating routine can include computer instructions configured to calculate or determine a plurality of cycles in a spinal decompression treatment profile. When performed by system 100, each cycle directs system 100 to apply a tensile force to a patient's spine at a high tension level for a high tension period of time and at a low tension level for a low tension period of time, as described herein. As described below, generating routine also may be employed to make on-the-fly calculations and/or determinations of a tensile force that is to be applied to a patient based on which portions of a treatment profile have been completed and/or which portion of the treatment profile is currently being applied.

The treatment cycle adjustment routine can include computer instructions configured to alter or modify any one or more of the high and low tension levels and the high and low tension periods of time. The adjustment routine can modify any part or whole of a cycle in the treatment profile in order to achieve any of the modifications described above. In addition, adjustment routine also may be employed to make on-the-fly calculations and/or determinations of a tensile force that is to be applied to a patient based on which portions of a treatment profile have been completed and/or which portion of the treatment profile is currently being applied.

The feedback routine can include computer instructions configured to measure at least one of an expected tensile force, an unexpected tensile force, and a corrective tensile force. As described above, dynamic smoothing can be applied to any expected or unexpected change in tensile force applied to a patient's spine. The expected tensile force can be communicated to feedback routine from generating routine and/or adjustment routine. The unexpected tensile force can include an actual tensile force applied to a patient's spine, as described above. The unexpected tensile force may be measured by control system 114, actuator 110, and/or a tensile feedback system 108 (described below). The feedback routine may then calculate a corrective tensile force by determining a difference between the expected and unexpected tensile forces. The corrective tensile force can then be communicated to actuator 110 and/or tensile feedback system 108 (described below). Once actuator 110 and/or tensile feedback system 108 receive the corrective tensile force, the corrective tensile force may be applied to the patient's spine.

Control system 114 may be controlled through a patient interface system 112. Interface system 112 includes any input device configured to allow a user of system 100 to regulate control system 114. For example, patient interface system 112 may include a touchscreen monitor. However, patient interface system 112 may include any form of data entry device. Patient interface system 112 may be controlled through a software and/or firmware application and may communicate with control system 114.

System 100 may also include a tensile feedback system 108 (not shown). Feedback system 108 may include a loadcell or dynamometer located within the region of system 100 where actuator 110 is located. Feedback system 108 may be located inline with actuator 110, so as to allow for feedback to control system 114. As described above, a feedback routine may communicate a corrective tensile force to feedback system 108 to be applied to a patient's spine.

In operation, a decompression treatment on a patient may begin by positioning the patient onto bed or table 102. The patient's head may be positioned at the head end 120 and the patient's feet may be positioned at the tail end 116. The patient may be outfitted with a patient harness device that allows for connection to patient interface device 104 and positions the tensile forces generated by actuator 110 in proper position(s) relative to the patient. The patient harness device may be connected to patient interface device 104 through any type of connection device, such as a clip or buckle that may alternatively secured and removed, for example.

A user of decompression system 100 (for example, a physician) may use patient interface system 112 to select one or more treatment parameters for a decompression therapy. As described above, treatment parameters for a decompression therapy include a high tension level or plateau, a low tension level plateau, a high tension time period, and a low tension time period. The user may select the treatment parameters individually in order to formulate one or more repeating cycles of a treatment profile, or may select a preconfigured treatment profile accessible by control system 114. Once the user has selected the parameters of the treatment profile or has selected a treatment profile, patient interface system 112 may communicate the parameters and/or treatment profile to control system 114.

Control system 114 then executes the treatment profile corresponding to the user's selected parameters and/or pre-configured treatment profile. Control system 114 may execute the treatment profile by activating actuator 110 and adjusting the tensile output of actuator 110 according to the tensile forces required by one or more cycles in the treatment profile entered by the user. The treatment profile may consist of low and high tension plateaus above 125 pounds, and may also consist of any of the decompression therapy variations described in the present invention, for example.

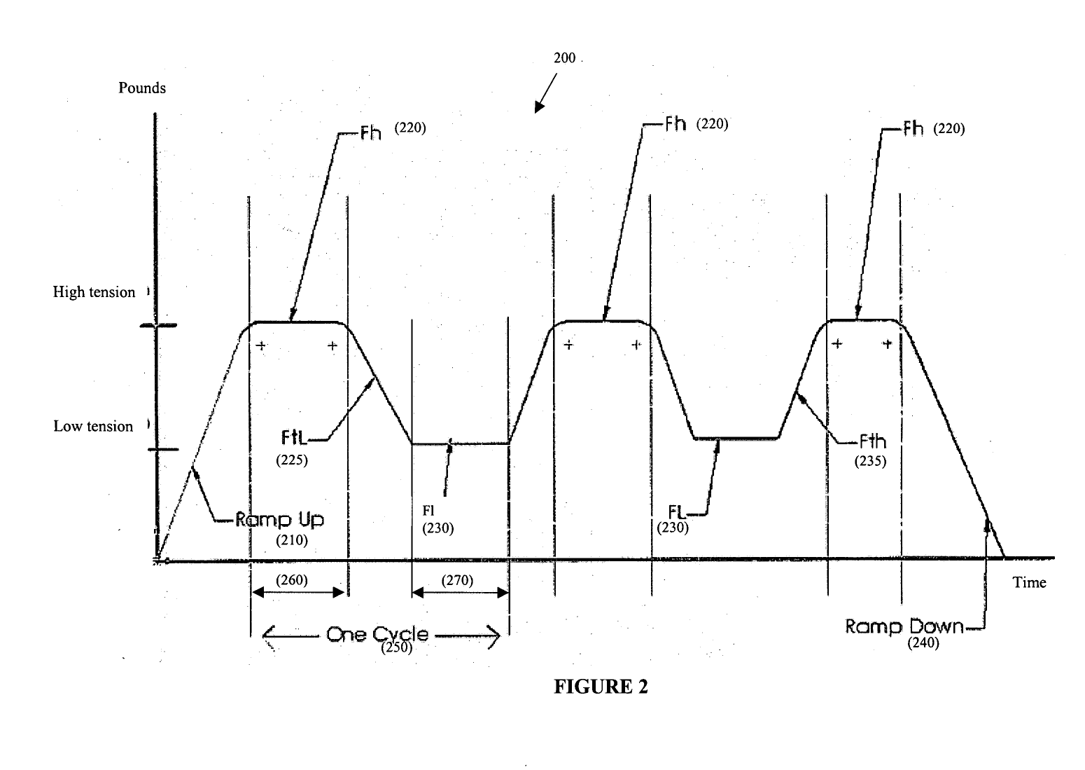

FIG. 2 illustrates an exemplary treatment profile 200 for spinal decompression therapy in accordance with an embodiment of the present invention. Treatment profile 200 may be created and/or selected by the user using interface system 112, as described above.

Treatment profile 200 may be continuously monitored by feedback system 108 for tensile force adjustment to any deviations from the tensile forces required by treatment profile 200. This monitoring allows for closely controlled decompression profiles. Treatment profile 200 includes several sections, namely a ramp up 210, a high tension level (“Fh”) 220, a low tension level (“FL”) 230, a tension adjust low (“FtL”) 225, a tension adjust high (“Fth”) 235, and ramp down 240.

Ramp up 210 includes a portion of profile 200 where a tensile force is increased from zero slowly upwards to high tension level 220 Ramp up 210 may be performed slowly and may be calculated and/or implemented using one or more of the family of non-linear, curvilinear, sinusoidal, exponential, or logarithmic mathematical functions. In other words, one or more of these functions may be used to describe the ramp up 210 function of tension with respect to time. These functions can provide a smooth transition into treatment profile 200 and may be used to taper the transition of tension from the ramp up 210 into high tension level 220. Such a taper can allow for a non-abrupt leveling off of the tension applied to a patient, for example.

The high tension level 220 section of profile 200 can be maintained for a period of time, such as a high tension time period 260. Next, profile 200 proceeds to tension adjust low 225 where profile 200 includes a tension transition from high tension level 220 to low tension level 230. The low tension level 230 section can be maintained for a period of time, such as a low tension time period 270.

The next portion of profile 200 includes tension adjust high 235. Tension adjust high 235 includes the transition from low tension level 230 to high tension level 220. Tension adjust high 235 differs from ramp up 210 as ramp up 210 includes a transition from no tension force to high tension level 220, while tension adjust high 235 includes a transition from low tension level 230 to high tension level 220.

Profile 200 may terminate with a ramp down 240 section. Ramp down 240 includes a portion of profile 200 where the tensile force is adjusted from any tension level (such as low tension level 230 or high tension level 220, as shown in FIG. 2) to zero force, or a tensile force at which profile 200 began.

A cycle for treatment profile 200 includes a time period encompassing one full high 220 and low 230 tension plateau. In other words, a cycle 250 of profile 200 can include an amount of time encompassing high tension level 220, tension adjust low 225, and low tension level 230. Therefore, an amount of time encompassed by cycle 250 can include high tension time period 260, low tension time period 270, and an amount of time over which tension adjust low 225 occurs. An exemplary treatment profile 200 may last between 30 and 45 minutes and may include between 18 and 30 cycles, for example.

In another embodiment of the present invention, a cycle 250 of profile 200 may be defined to encompass an amount of time that includes high tension level 220, tension adjust low 225, low tension level 230, and tension adjust high 235. Therefore, an amount of time encompassed by cycle 250 in such an embodiment includes high tension time period 260, low tension time period 270, an amount of time over which tension adjust low 225 occurs, and an amount of time over which tension adjust high 235 occurs.

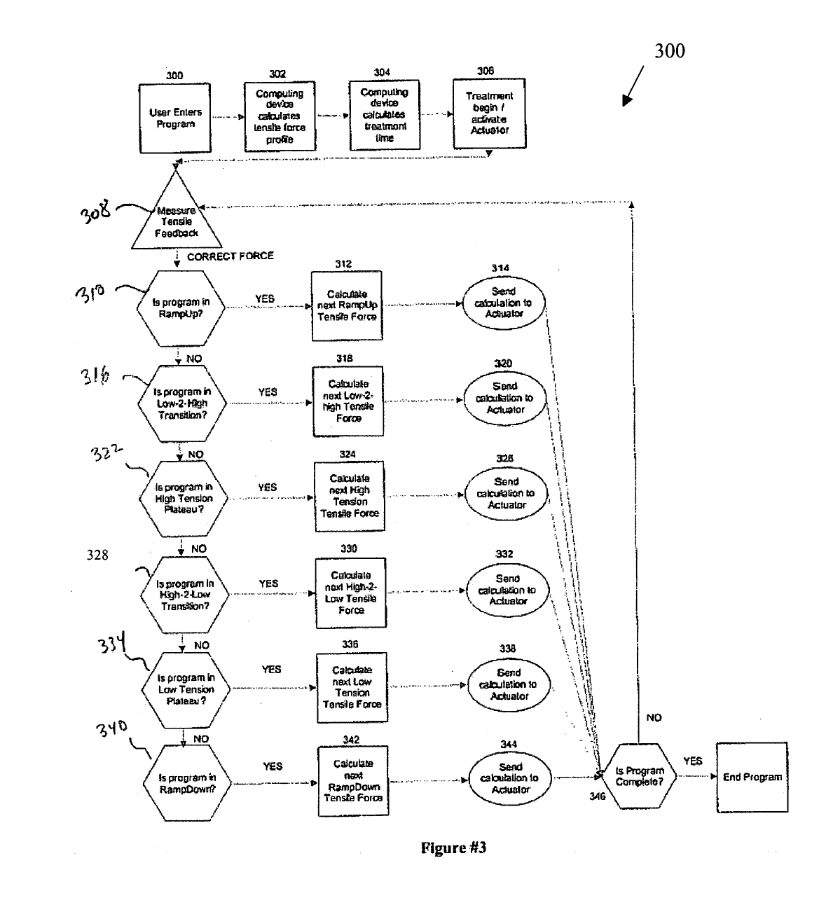

FIG. 3 includes a flowchart for a method 300 of implementing a spinal decompression profile in accordance with an embodiment of the present invention. Method 300 begins with at step 301, where a user selects one or more parameters of a treatment profile and/or a predetermined treatment profile. As described above, a user may employ interface 112 to select the parameters and/or treatment profile. The parameters and/or treatment profile is(are) then communicated to control system 114.

Next, at step 302, control system 114 calculates a tensile force profile. A tensile force profile includes the tension forces to be applied over one or more cycles of a treatment profile. For example, a tensile force profile may include ramp up 210, high 220 and low 230 tension profiles, high 235 and low 225 tension adjust, and ramp down 240 of a treatment profile 200 as illustrated in FIG. 2. Control system 114 may calculate the tensile force profile by using a spinal decompression treatment profile generating routine, as described above.

Next, at step 304, control system 114 calculates a treatment time. The treatment time can include any one or more of an amount of time encompassed by a cycle of a treatment profile, an amount of time encompassed by an entire treatment profile, or an amount of time encompassed by a portion of a treatment profile. Control system 114 may calculate the treatment time by using the spinal decompression treatment profile generating routine.

Next, at step 306, control system 114 communicates with actuator 110 in order to activate actuator 110 for the treatment profile. For example, control system 114 may communicate an initial tensile force to be applied to a patient.

Next, at step 308, feedback device 108 measures an amount of actual tensile force applied to a patient's spine, as described above. Feedback device 108 can communicate this measurement to control system 314. Control system 314 may then determine a tensile force difference, as described above. Also as described above, control system 114 may determine the tensile force difference by employing a feedback routine. At this time, control system 114 can correct for the difference between the expected and measured tensile forces.

Next, a determination is made as to which section of the treatment profile is currently being applied. In other words, an assessment is made as to which section of the treatment profile is completed and which section is next. At step 310, if the treatment profile is in ramp up 210, then method 300 proceeds to step 312 where next tensile force is calculated. For example, control system 114 may determine at step 310 that the treatment profile is still in ramp up 210. Therefore, at step 312, control system 114 determines the next tensile force to be applied in continuing with ramp up 210. As described above, any one or more of generating routine and adjustment routine of control system 114 may determine the next tensile force to be applied. Next, at step 314, the calculated or determined tensile force is communicated to actuator 1 10. Actuator 110 may then apply the tensile force to the patient's spine.

If the treatment profile is determined to not be in ramp up 210 at step 310, method 300 proceeds to step 316, where it is determined if the treatment profile is in tension adjust high 235. If the treatment profile is in tension adjust high 235, then the next tensile force is calculated at step 318. The tensile force is communicated to actuator 110 at step 320 and is applied to the patient.

If the treatment profile is determined to not be in tension transition high 235 at step 316, method 300 proceeds to step 322, where it is determined if the treatment profile is in high tension level 220. If the treatment profile is in high tension level 220, then the next high tensile force is calculated at step 324. The tensile force is communicated to actuator 110 at step 326 and is applied to the patient.

If the treatment profile is determined to not be in high tension level 220 at step 322, method 300 proceeds to step 328, where it is determined if the treatment profile is in tension adjust low 225 if the treatment profile is in tension adjust low 225, then the next tensile force is calculated at step 330. The tensile force is communicated to actuator 110 at step 332 and is applied to the patient.

If the treatment profile is determined to not be in tension adjust low 225 at step 328, method 300 proceeds to step 334, where it is determined if the treatment profile is in low tension level 230. If the treatment profile is in low tension level 230, then the next low tensile level force is calculated at step 336. The tensile force is communicated to actuator 110 at step 338 and is then applied to the patient.

If the treatment profile is determined to not be in low tension level at step 334, method 300 proceeds to step 340, where it is determined if the treatment profile is in ramp down 240. If the treatment profile is in ramp down 240, then the next tensile force is calculated at step 342. The tensile force is communicated to actuator 110 at step 344 and is then applied to the patient.

After any one or more of steps 314, 320, 326, 332, 338, and 344, method 300 proceeds to step 346 where a determination is made as to whether the treatment profile has been completed. If the profile has been completed, method 300 terminates. If the profile has not been completed, then method 300 proceeds back to step 308. In other words, while a treatment profile is ongoing, method 300 may continue in a loop-wise fashion through the various steps of determining which portion of a treatment profile is ongoing.

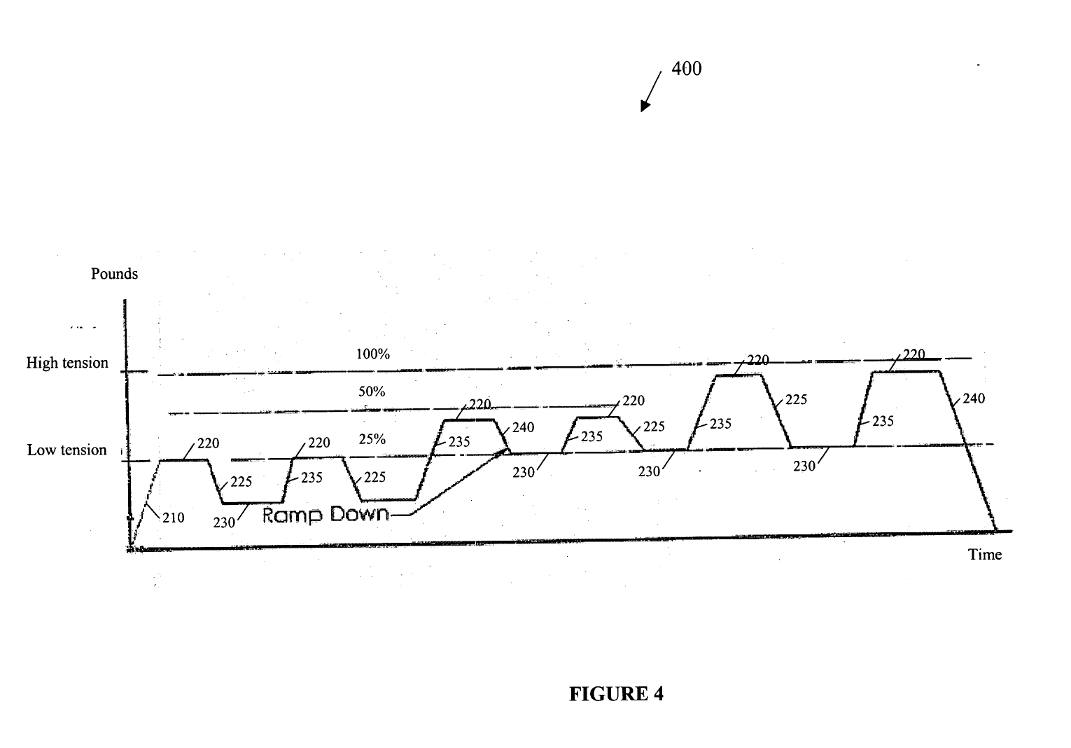

As described above, various embodiments of the present invention provide for modifications to traditional spinal decompression therapies. FIG. 4 illustrates an exemplary spinal decompression treatment profile 400 that includes the modification of a gradual treatment profile in accordance with an embodiment of the present invention. As described above, treatment profile 400 may be continuously monitored for tensile force adjustment allowing for closely controlled decompression profiles.

Profile 400 is described by various sections such as ramp up 210, high tension level 220, tension adjust low 225, low tension level 230, tension adjust high 235, and ramp down 240, each as described above.

Profile 400 is similar to profile 200 with a modification. The modification includes the multiplication of one or more variables or parameters by high tension level 220, low tension level 230, a maximum high tension level, and a maximum low tension level for at least one cycle. The variable(s) or parameter(s) may be user-defined. For example, the variables may be defined as a part or percentage of a maximum high tension level and/or low tension level.

For example, in profile 400, each cycle of alternating high 220 and low 220 tension levels is multiplied by a percentage of a maximum high tension level and a maximum low tension level, respectively. In the example of FIG. 4, in the first two cycles, the high 220 and low 230 tension levels are each multiplied by 25% of the maximum high tension level and maximum low tension level, respectively, to yield ¼ of the maximum high and low tension levels. In the next two cycles, the high 220 and low 230 tension levels are each multiplied by 50% of the maximum high tension level and maximum low tension level, respectively, to yield ½ of the maximum high and low tension levels. In the remaining cycles, the high 220 and low 230 tension levels are each multiplied by 100% of the maximum high tension level and maximum low tension level, respectively, so that the high tension level is equivalent to the maximum high tension level and the low tension level is equivalent to the maximum low tension level.

Typical treatment profiles may last between 30 and 45 minutes and may include between 18 and 30 cycles. Once the treatment profile reaches the 100% cycle multiplier section, it may continue at the 100% level until treatment profile concludes.

In another embodiment of the present invention, the variable or parameter used to modify a treatment profile according to gradual treatment profile may act on only one of the high 220 and low 230 tension levels. For example, the low tension level 230 may remain constant throughout all cycles of the treatment profile while the high tension level 220 begins as a fraction of the maximum high tension level and increases with each cycle.

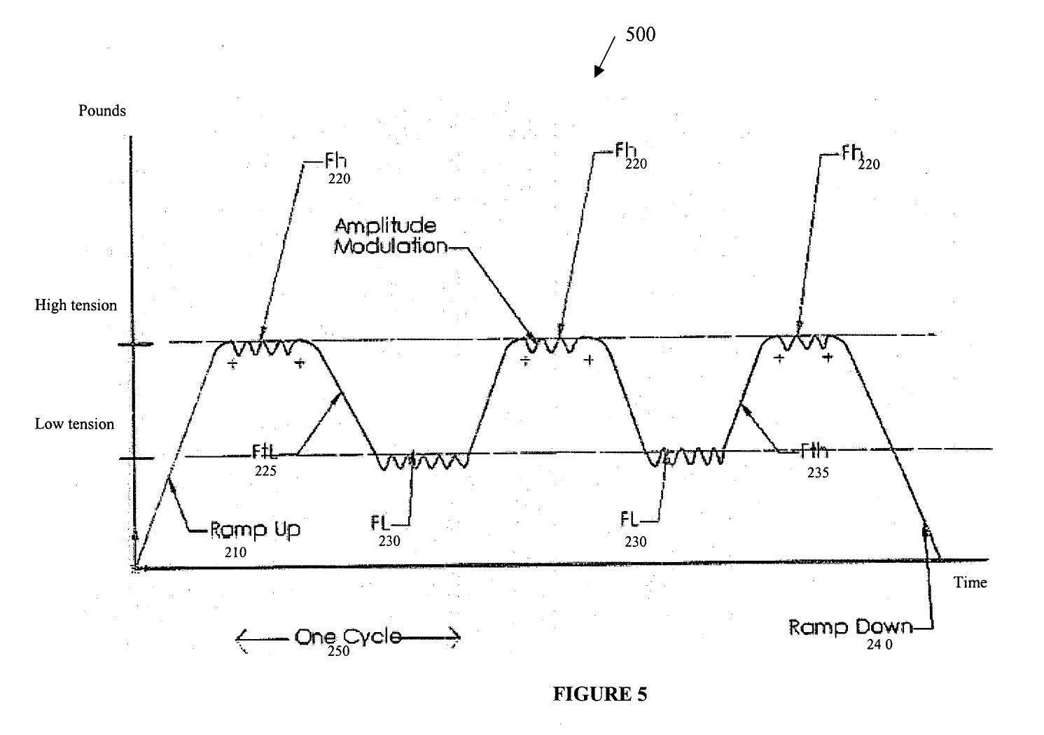

FIG. 5 illustrates an exemplary spinal decompression treatment profile 500 that includes the modification of tension amplitude modulation in accordance with an embodiment of the present invention. As described above, treatment profile 500 may be continuously monitored for tensile force adjustment allowing for closely controlled decompression profiles.

Tension amplitude modulation includes the modulation of a tension level with respect to time. In other words, one or more of high tension level 220 and low tension level 230 may be varied with respect to time over high tension time period 260 and low tension time period 270, respectively. For example, in FIG. 5, each of high and low tension levels 220, 230 are modulated using a sinusoidal function. However, any mathematical function may be used to modulate one or more tension levels 220, 230.

The amplitude modulation modification can be applied to only high tension level 220, low tension level 230, or for both high and low tension levels 220, 230. The amplitude modulation may also occur at a user-selected tension level 220 or 230 and can last for the entire duration of that level, or a portion thereof, throughout the treatment program.

The frequency of the amplitude modulation can also be user defined. For example, a user may define a rate of amplitude modulation with respect to time for one or more cycles in treatment profile 500.

The amount of amplitude modulation may also be user defined. Control system 114 may include limitations on the amount of amplitude modulation. For example, control system 114 may limit the amount of amplitude modulation to a range bounded by high and low tension levels 220, 230. In addition, the amount of amplitude modulation may be subtracted from high and low tension levels 220, 230, so as not to exceed a user's maximum defined settings or high and low tension levels 220, 230, for example.

Typical treatment profiles may last between 30 and 45 minutes and may consist of between 18 and 30 cycles, for example. Amplitude modulation may continue through an entire treatment profile or only during user-defined sections of the treatment profile. Amplitude modulation may be applied to any part of the treatment profile including ramp up 210, ramp down 240 and any of the transition sections (such as 225 and 235, for example). Amplitude modulation may increase the confusion and relaxation of the paraspinal muscles, which aids in the distraction of spinal vertebra and/or discs.

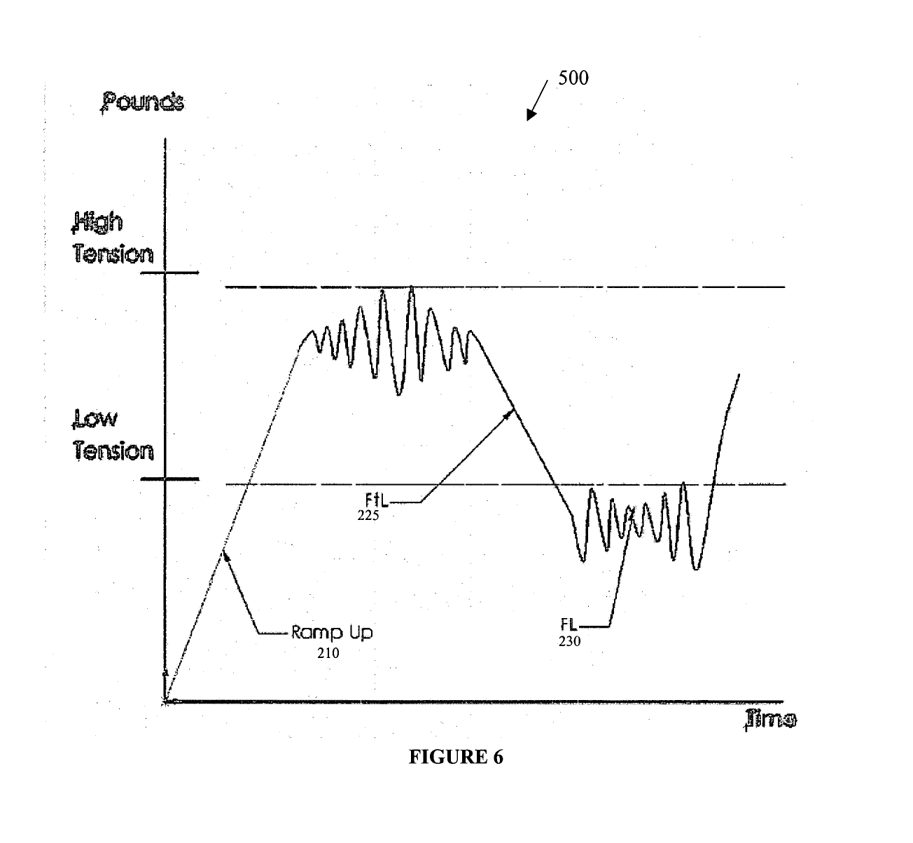

FIG. 6 illustrates an exemplary cycle of spinal decompression treatment profile 500 with the further modification of varying an amount of tension amplitude modulation in accordance with an embodiment of the present invention. As described above, treatment profile 500 may be continuously monitored for tensile force adjustment allowing for closely controlled decompression profiles.

Treatment profile 500 is further modified in FIG. 6 by the multiplication of one or more user-defined variables by the amount of amplitude modulation for high tension level 220 and low tension level 230. For example, a user may select a maximum range or percentage for the amplitude modulation. Control system 114 may then control actuator 110 so as to vary the amount of modulation with respect to time over one or more of high and low tension time periods 260, 270. For example, in the exemplary cycle illustrated in FIG. 6, the tension modulation over high tension time period 260 begins at a very small amount, increases to a maximum amount, and then decreases to a small amount. In contrast, tension modulation over low tension time period 270 begins at a maximum amount and decreases to a minimum before increasing back to the maximum.

This modification may be applied for one or more high tension levels 220 alone, one or more low tension levels 230 alone, or both high 220 and low 230 tension levels. The tension levels 220, 230 (the tension levels where amplitude modulation can occur) may be selected by a user. The modulation may be applied to an entire high 260 or low 270 tension time period or any portion thereof. In addition, the modulation may be applied for a portion of a cycle, an entire cycle, a portion of a treatment profile, or for an entire treatment profile.

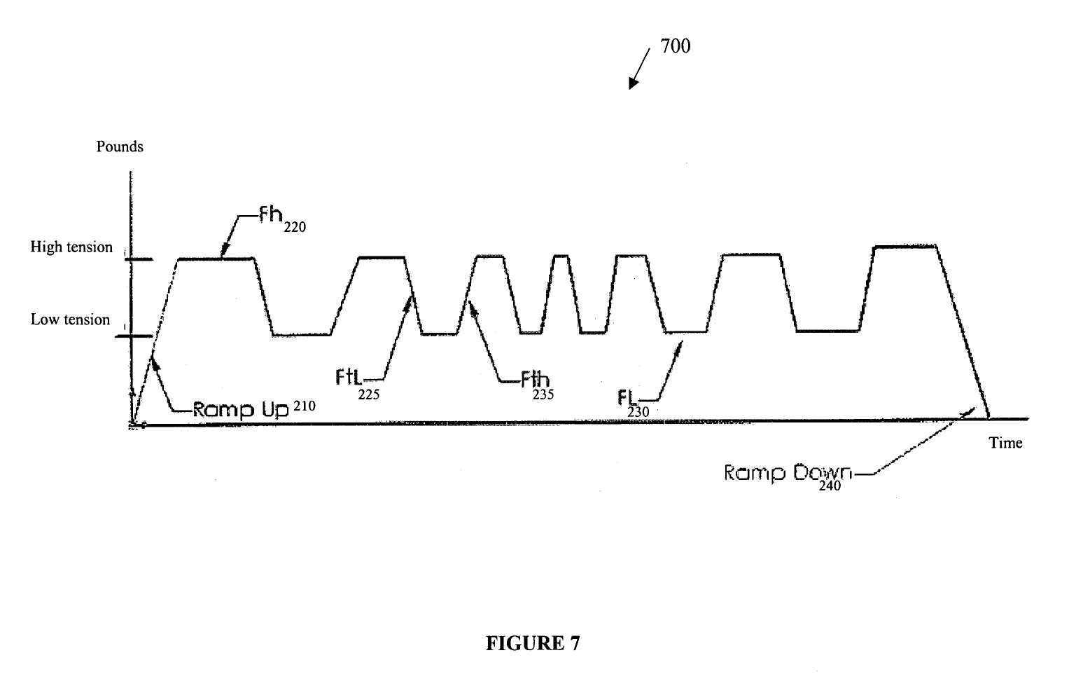

FIG. 7 illustrates an exemplary spinal decompression treatment profile 700 that includes the modification of frequency modulation of high 260 and low 270 tensile time periods in accordance with an embodiment of the present invention. As described above, treatment profile 700 may be continuously monitored for tensile force adjustment allowing for closely controlled decompression profiles.

Profile 700 is further modified by the multiplication of one or more user definable variables that determine one or more frequency modulations of high 260 and/or low 270 tension time periods of profile 700. In general, a frequency modulation of high 260 and/or low 270 tension time periods includes varying an amount of time spent at high tension level 220 and/or low tension level 230, respectively, with respect to a number of cycles. For example, during a first cycle, a high tension time period 260 may differ from a high tension time period 260 during a second cycle.

Any number of parameters can be defined by a user to achieve frequency modulation of high 260 and low 270 tension time periods. For example, a user can define a maximum or minimum frequency, high tension time period 260 and/or low tension time period 270. In such an example, control system 114 can gradually alter the length of high 260 and/or low 270 time periods for each cycle in temporally sequential cycles until the user-defined maximum or minimum frequency, high tension time period 260 and/or low tension time period 270 is achieved.

In another example, a user can define an amount that the frequency, high tension time period 260 and/or low tension time period 270 is to be adjusted between cycles. In such an example, control system 114 may adjust the frequency, high tension time period 260 and/or low tension time period 270 for each cycle by changing the frequency, high 260 and/or low 270 tension time period by the user defined amount until a user- or system-defined minimum or maximum frequency, high 260 and/or low 270 tension time period is achieved.

Profile 700, for example, includes a frequency modulation that causes high 260 and low 270 tension time periods to be longer in the first cycle and gradually decrease as the number of cycles increases. However, the frequency of the time period modulation reaches a maximum (i.e., the high 260 and low 270 tension time periods reach a minimum) at the fourth cycle of profile 700. After the fourth cycle, high 260 and low 270 time periods for subsequent cycles gradually increase back to the original frequency of the first cycle of profile 700.

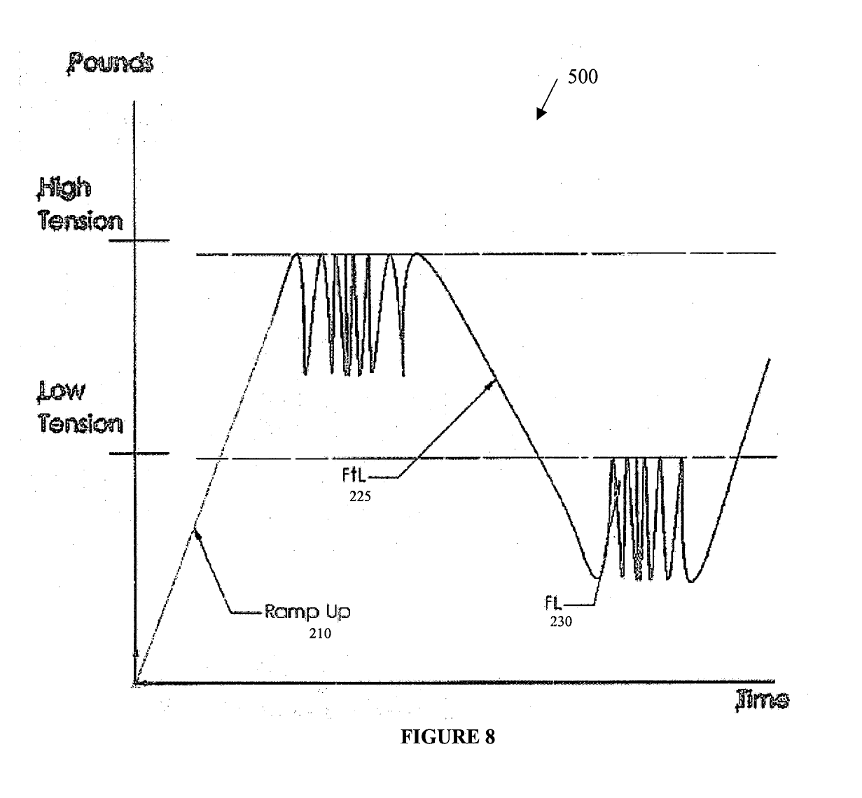

FIG. 8 illustrates an exemplary cycle of spinal decompression treatment profile 500 that includes the modification of frequency modulation of tension amplitude modulation in accordance with an embodiment of the present invention. As described above, treatment profile 500 may be continuously monitored for tensile force adjustment allowing for closely controlled decompression profiles.

As described above, profile 500 includes the modulation of an amount of tension amplitude over one or more of high 220 and low 230 tension levels. A user may further modify profile 500 to include the modulation of a frequency of the amplitude modulation. In other words, the rate at which the tension is being varied with respect to time during amplitude modulation may be further altered, or modulated. The effect of frequency modulation on amplitude modulation is to increase and/or decrease the rate or frequency of the amplitude modulation during one or more of high 220 and low 230 tension levels.

A user may choose to apply frequency modulation to the amplitude modulation for only high tension levels 220, low tension levels 230, or for both high 220 and low 230 tension levels. In addition, a user may select to apply frequency modulation to the amplitude modulation for selected cycles or for a selected number of temporally adjacent cycles.

A user may also choose to apply frequency modulation to the amplitude modulation for an entire high 260 or low 270 tension time period or any portion thereof. Similarly, a user may also choose to apply frequency modulation to the amplitude modulation for an entire cycle of profile 500 or any portion thereof.

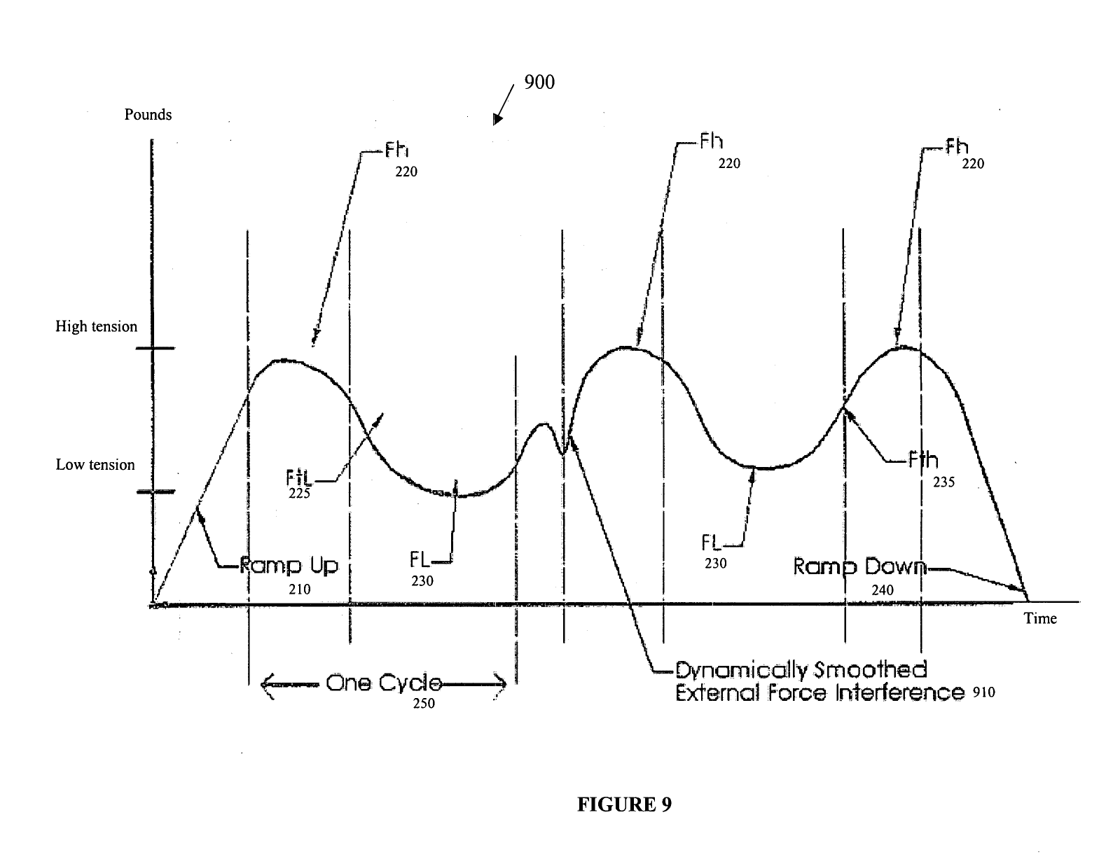

FIG. 9 illustrates an exemplary spinal decompression treatment profile 900 that includes the modification of dynamic smoothing in accordance with an embodiment of the present invention. As described above, treatment profile 700 may be continuously monitored for tensile force adjustment by feedback device 108, thereby allowing for closely controlled decompression profiles. Also as described above, a feedback routine may calculate an adjustment to a tensile force applied to a patient.

Dynamic smoothing is a modification to a treatment profile that provides more gradual, sloped progressions between tension levels and varies the slope of the high 220 and low 230 tension levels. Dynamic smoothing may be implemented by calculating aggressive slope variables for the transitions between tension levels. The aggressive slope variables may be defined by one or more functions from the family of non-linear, curvilinear, sinusoidal, exponential, or logarithmic mathematical functions, for example. In other words, the dynamic smoothing modification can reduce or eliminate abrupt changes in slope between (1) ramp up 210 and high tension level 210, (2) high tension level 220 and tension adjust low 225, (3) tension adjust low 225 and low tension level 230, (4) low tension level 230 and tension adjust high 235, and (5) high tension level 220 and ramp down 240, for example. By smoothing the transitions between two portions of a cycle or between cycles in a treatment profile, paraspinal muscle opposition is lowered to the decompression treatment.

Dynamic smoothing can also apply to tensile force corrections. As described above, a feedback routine of control system 114 can measure a difference between a tensile force that is expected to be applied to a patient and a tensile force that is actually applied to the patient. When an external force acts on the decompression system (such that it alters the intended tensile loading profile), control system 114 may correct for that external force. This correction can take any form of an immediate ramp up or down to the intended tensile force.

Dynamic smoothing seeks to react quickly to smooth the effect of the external force. Control system 114 can calculate tensile force corrections and direct actuator 110 to apply corrective forces such that they take the form of those from any of the family of non-linear, curvilinear, sinusoidal, exponential, or logarithmic mathematical functions. For example, at point 910 of FIG. 9, control system 114 has reacted to an unexpected external force. Feedback routine in control system 114 may calculate a correcting tensile force and communicate it to actuator 110 in a manner that provides for a smooth transition when the correcting force is applied, as illustrated by the smooth transition in tensile forces both before and after point 910 of FIG. 9.

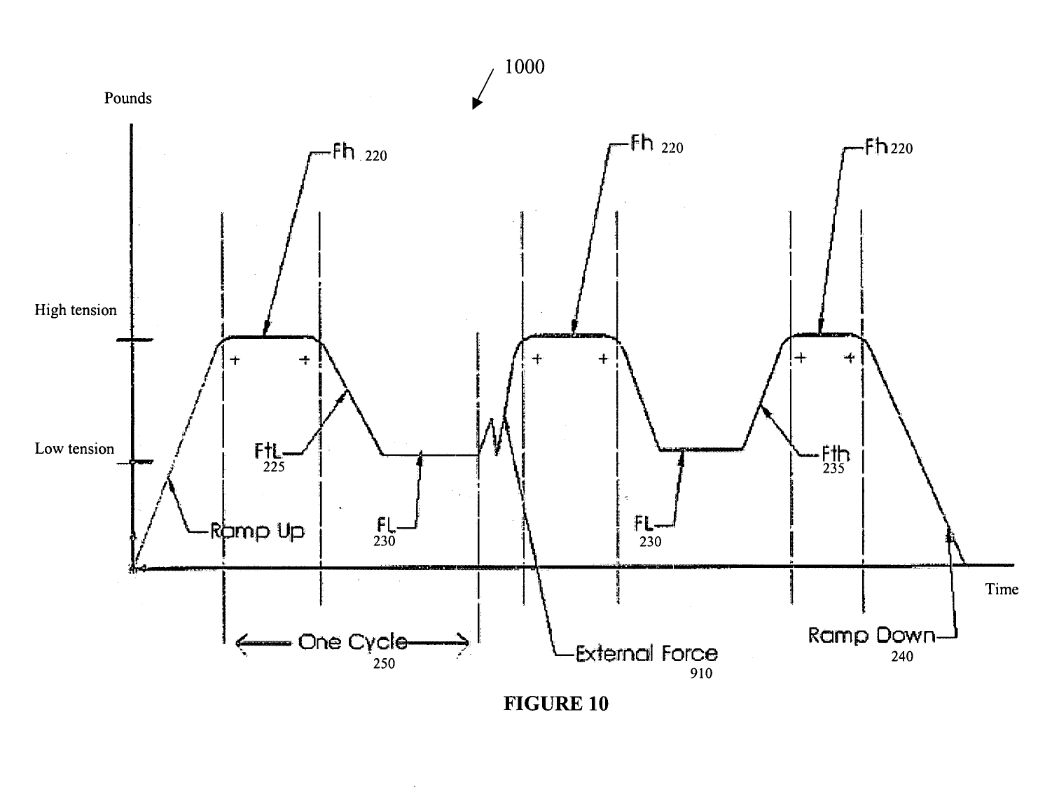

In another embodiment of the present invention, control system 114 does not apply dynamic smoothing to correct for an unexpected external force. FIG. 10 illustrates a treatment profile 1000 applying a corrective tensile force without dynamic smoothing in accordance with an embodiment of the present invention. As described above, control system 114 may measure a difference between an expected and actual tensile force applied to a patient. Control system 114 may then direct actuator 110 to apply a tensile force sufficient to correct for the difference between expected and actual tensile forces. As illustrated in FIG. 1000, without dynamic smoothing, the corrective force applied by actuator 110 may result in abrupt changes in tensile forces before and after point 910 (the point at which the corrective tensile force is applied).

Any one or more of the previously described modifications may be calculated or determined by a treatment cycle adjustment routine. The adjustment routine may be embodied in a set of instructions stored on computer-readable medium, such as a computer program. The adjustment routine may calculate or determine any one or more of the previously described modifications for a single cycle in a treatment profile, or for all cycles in a treatment profile. In general, the profile generating routine creates a treatment profile based on user input while the adjustment routine determines modifications to the treatment profile.

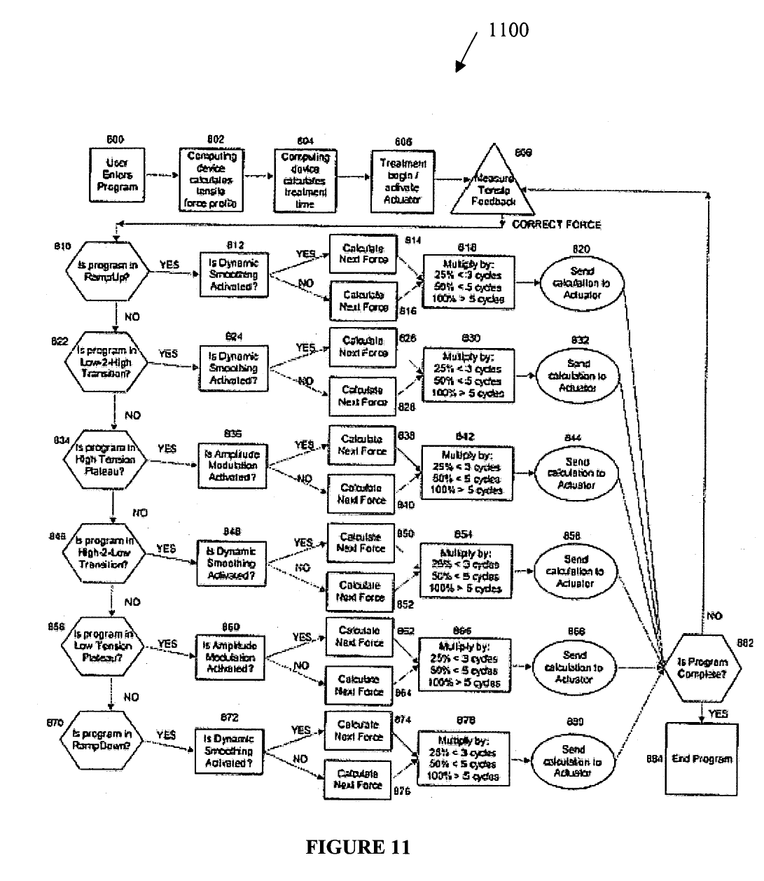

FIG. 11 illustrates a method 1100 for adjusting a treatment profile in accordance with an embodiment of the present invention. Method 1100 may be employed by the generating and adjustment routines to create and adjust a treatment profile. However, method 1100 is intended merely as an example, and not a limitation on the functionality of the generating and adjustment routines. For example, while method 1100 does not address frequency modulation, such a modification may be used by the adjustment routine to modify a treatment profile. In addition, while method 1100 assumes that the treatment profile has been modified with a gradual increase in high 220 and/or low 230 tension levels, such a modification is not necessarily present in all embodiments of the present invention.

Method 1100 begins at step 800, where a user selects one or more parameters and/or a predetermined treatment profile, as described above. As described above, a user may employ interface 112 to select the parameters and/or treatment profile. The parameters and/or treatment profile is(are) then communicated to the generating routine of control system 114.

Next, at step 802, the generating routine calculates a tensile force profile based on the parameter(s) and/or profile(s) selected by the user.

Next, at step 804, the generating routine calculates a treatment time, as described above.

Next, at step 806, control system 114 communicates with actuator 110 in order to activate actuator 110 for the treatment profile. For example, control system 114 may communicate an initial tensile force to be applied to a patient.

Next, at step 808, feedback device 108 measures an amount of actual tensile force applied to a patient's spine, as described above. Feedback device 108 can communicate this measurement to control system 314. The feedback routine may then determine a tensile force difference, as described above. Control system 114 can then correct for the difference between the expected and measured tensile forces.

Next, a determination is made as to which section of the treatment profile is currently being applied. In other words, an assessment is made as to which section of the treatment profile is completed and which section is next. At step 810, if the treatment profile is in ramp up 210, then method 1100 proceeds to step 812.

At step 812, a determination is made as to whether dynamic smoothing has been activated for the treatment profile. Dynamic smoothing (or any other modification to a treatment profile) may be activated when a user selects the modification during step 800. If dynamic smoothing has been activated, method 1100 proceeds to step 814, where the adjustment routine calculates a dynamically smoothed application of a tensile force to be applied. As described above, an application of a tensile force may be dynamically smoothed by smoothing calculating aggressive slope variables for the transitions between tension levels. The aggressive slope variables may be defined by one or more functions from the family of non-linear, curvilinear, sinusoidal, exponential, or logarithmic mathematical functions, for example.

If it is determined at step 812 that dynamic smoothing is not activated, then method 1100 proceeds to step 816 where the adjustment routine calculates a non-dynamically smoothed application of a tensile force to be applied.

After step 814 or 816, method 1100 proceeds to step 818, where the adjustment routine calculates the next tensile force level. As described above, method 1100 illustrates the modification of a treatment profile that includes gradual increases in calculated tensile forces. The gradual increases in tensile forces are determined by adjustment routine at step 818. In the embodiment of the present invention illustrated in method 1100, the gradual increase in tensile forces includes multiplying the tensile force calculated in step 814 or 816 (1) by 25% during the first three cycles 250 of the treatment profile, (2) by 50% during the fourth and fifth cycles 250 of the treatment profile, and (3) by 100% during all cycles 250 of the treatment profile after the fifth cycle 250. Therefore, the tensile force calculated by the adjustment routine is further modified by multiplying the tensile force by a factor that is based on the number of cycles 250 that have been applied.

Next, at step 820, the newly calculated tensile force is communicated from adjustment routine of control system 114 to actuator 110. Actuator 110 may then apply a tensile force to the patient, as described above.

However, if after step 808 it is determined that the treatment profile is not in ramp up 210, then at step 822 a determination is made as to whether the treatment profile is in tension adjust high 235. If, at step 822, it is determined that the treatment profile is in tension adjust high 235, then method 1100 proceeds to step 824.

At step 824, a determination is made as to whether dynamic smoothing has been activated for the treatment profile. Dynamic smoothing (or any other modification to a treatment profile) may be activated when a user selects the modification during step 800. If dynamic smoothing has been activated, method 1100 proceeds to step 826, where the adjustment routine calculates a dynamically smoothed application of a tensile force to be applied. As described above, an application of a tensile force may be dynamically smoothed by smoothing calculating aggressive slope variables for the transitions between tension levels. The aggressive slope variables may be defined by one or more functions from the family of non-linear, curvilinear, sinusoidal, exponential, or logarithmic mathematical functions, for example.

If it is determined at step 824 that dynamic smoothing is not activated, then method 1100 proceeds to step 828 where the adjustment routine calculates a non-dynamically smoothed application of a tensile force to be applied.

After step 824 or 828, method 1100 proceeds to step 830, where the adjustment routine calculates the next tensile force level. As described above, method 1100 illustrates the modification of a treatment profile that includes gradual increases in calculated tensile forces. Therefore, the tensile force calculated by the adjustment routine is further modified by multiplying the tensile force by a factor that is based on the number of cycles 250 that have been applied, as described above.

Next, at step 832, the newly calculated tensile force is communicated from adjustment routine of control system 114 to actuator 110. For example, adjustment routine may communicate the next tensile force to be applied to a patient and/or one or more of the newly calculated high 220 and/or low 230 tension levels to actuator 110. Actuator 110 may then apply a tensile force to the patient, as described above. The tensile force applied by actuator 110 need not be equivalent to high 220 or low 230 tension level. As described above, during a treatment profile, actuator 110 applies tensile forces that vary between high 220 and low 230 tension levels at various times, including, for example, during ramp up 210, tension adjust low 225, and tension adjust high 235. During any one of these periods, the tensile force applied by actuator 110 may be at high 220 or low 230 tension level, or any tension between high 220 and low 230 tension levels.

However, if after step 808 it is determined that the treatment profile is not in ramp up 210 or tension adjust high 235, then at step 834 a determination is made as to whether the treatment profile is in high tension level 220. If, at step 834, it is determined that the treatment profile is in high tension level 220, then method 1 100 proceeds to step 836.

At step 836, a determination is made as to whether amplitude modulation has been activated for high tension level 220 in the treatment profile. Amplitude modulation for high 220 and/or low 230 tension levels (or any other modification to a treatment profile) may be activated when a user selects the modification(s) during step 800. If amplitude modulation has been activated, method 1100 proceeds to step 838, where the adjustment routine calculates a tensile force (modified by amplitude modulation) to be applied, as described above. For example, if method 1100 includes amplitude modulation of high 220 tension level by employing a sinusoidal function, then a tensile force that is a sinusoidal variation of high 220 tension level is calculated.

If it is determined at step 836 that amplitude modulation is not activated, then method 1100 proceeds to step 840 where the adjustment routine calculates a tensile force (that is not modified by amplitude modulation) to be applied.

After step 838 or 840, method 1100 proceeds to step 842, where the adjustment routine calculates the next tensile force level. As described above, method 1100 illustrates the modification of a treatment profile that includes gradual increases in calculated tensile forces. Therefore, the tensile force calculated by the adjustment routine is further modified by multiplying the tensile force by a factor that is based on the number of cycles 250 that have been applied, as described above.

Next, at step 844, the newly calculated tensile force is communicated from adjustment routine of control system 114 to actuator 110. For example, adjustment routine may communicate the next tensile force to be applied to a patient and/or one or more of the newly calculated high 220 and/or low 230 tension levels to actuator 110. Actuator 110 may then apply a tensile force to a patient, as described above. The tensile force applied by actuator 110 need not be equivalent to high 220 or low 230 tension level. As described above, during a treatment profile, actuator 110 applies tensile forces that vary between high 220 and low 230 tension levels at various times, including, for example, during ramp up 210, tension adjust low 225, and tension adjust high 235. During any one of these periods, the tensile force applied by actuator 110 may be at high 220 or low 230 tension level, or any tension between high 220 and low 230 tension levels.

However, if after step 808 it is determined that the treatment profile is not in ramp up 210, tension adjust high 235, or high tension level 220, then at step 846 a determination is made as to whether the treatment profile is in tension adjust low 225. If, at step 846, it is determined that the treatment profile is in tension adjust low 225, then method 1100 proceeds to step 848.

At step 848, a determination is made as to whether dynamic smoothing has been activated for the treatment profile. Dynamic smoothing (or any other modification to a treatment profile) may be activated when a user selects the modification during step 800. If dynamic smoothing has been activated, method 1100 proceeds to step 850, where the adjustment routine calculates a dynamically smoothed application of a tensile force to be applied.

If it is determined at step 848 that dynamic smoothing is not activated, then method 1100 proceeds to step 852 where the adjustment routine calculates a non-dynamically smoothed application of a tensile force to be applied.

After step 850 or 852, method 1100 proceeds to step 854, where the adjustment routine calculates the next tensile force level. As described above, method 1100 illustrates the modification of a treatment profile that includes gradual increases in calculated tensile forces. Therefore, the tensile force calculated by the adjustment routine is further modified by multiplying the tensile force by a factor that is based on the number of cycles 250 that have been applied, as described above.

Next, at step 856, the newly calculated tensile force is communicated from adjustment routine of control system 114 to actuator 110, as described above.

However, if after step 808 it is determined that the treatment profile is not in ramp up 210, tension adjust high 235, high tension level 220, or tension adjust low 225, then at step 858 a determination is made as to whether the treatment profile is in low tension level 230. If, at step 858, it is determined that the treatment profile is in low tension level 230, then method 1100 proceeds to step 860.

At step 860, a determination is made as to whether amplitude modulation has been activated for low tension level 230 in the treatment profile. Amplitude modulation for high 220 and/or low 230 tension levels (or any other modification to a treatment profile) may be activated when a user selects the modification(s) during step 800. If amplitude modulation has been activated, method 1100 proceeds to step 862, where the adjustment routine calculates a tensile force (modified by amplitude modulation) to be applied, as described above.

If it is determined at step 860 that amplitude modulation is not activated, then method 1100 proceeds to step 864 where the adjustment routine calculates a tensile force (that is not modified by amplitude modulation) to be applied.

After step 862 or 864, method 1100 proceeds to step 866, where the adjustment routine calculates the next tensile force level. As described above, method 1100 illustrates the modification of a treatment profile that includes gradual increases in calculated tensile forces. Therefore, the tensile force calculated by the adjustment routine is further modified by multiplying the tensile force by a factor that is based on the number of cycles 250 that have been applied, as described above.

Next, at step 868, the newly calculated tensile force is communicated from adjustment routine of control system 114 to actuator 110. For example, adjustment routine may communicate the next tensile force to be applied to a patient to actuator 110.

However, if after step 808 it is determined that the treatment profile is not in ramp up 210, tension adjust high 235, high tension level 220, tension adjust low 225, or low tension level 230, then at step 870 a determination is made as to whether the treatment profile is in ramp down 240. If, at step 870, it is determined that the treatment profile is in ramp down 240, then method 1100 proceeds to step 872.

At step 872, a determination is made as to whether dynamic smoothing has been activated for the treatment profile. If dynamic smoothing has been activated, method 1100 proceeds to step 874, where the adjustment routine calculates a dynamically smoothed application of a tensile force to be applied, as described above.

If it is determined at step 872 that dynamic smoothing is not activated, then method 1100 proceeds to step 876 where the adjustment routine calculates a non-dynamically smoothed application of a tensile force to be applied.

After step 874 or 876, method 1100 proceeds to step 878, where the adjustment routine calculates the next tensile force level. As described above, method 1100 illustrates the modification of a treatment profile that includes gradual increases in calculated tensile forces. The gradual increases in tensile forces are determined by adjustment routine at step 878, as described above.

Next, at step 880, the newly calculated tensile force is communicated from adjustment routine of control system 114 to actuator 110. Actuator 110 may then apply a tensile force to the patient, as described above.

After any of steps 820, 832, 844, 856, 868 and 880 (where newly calculated tensile forces are communicated to actuator 110), method 1100 proceeds to step 882 where a determination is made as to whether the treatment profile is complete. In other words, after a newly calculated tensile force is communicated to actuator 110, the adjustment routine of control system 114 determines if any additional tensile forces need to be calculated, or if the treatment profile is terminating. If it is determined that the treatment profile is complete, method 1100 proceeds from step 882 to step 884, where the treatment profile is terminated.

On the other hand, if it is determined that the treatment profile is not complete, then method 1100 proceeds after step 882 to step 808, where the feedback routine of control system 114 measures a tensile feedback, as described above.

Method 1100 therefore provides an example of a method for applying one or more modifications to a treatment profile in spinal decompression therapy. As described above, while method 1100 describes some modifications to a treatment profile, the present invention contemplates any combination of one or more modifications to a treatment profile described above.

While particular elements, embodiments and applications of the present invention have been shown and described, it is understood that the invention is not limited thereto since modifications may be made by those skilled in the art, particularly in light of the foregoing teaching. It is therefore contemplated by the appended claims to cover such modifications and incorporate those features that come within the spirit and scope of the invention.

What is claimed is:

1. A method of modifying a treatment profile in spinal decompression therapy, said method including:
applying a plurality of cycles of said treatment profile, each of said cycles including an application of a tensile force at a high tension level for a high tension time period and an application of said tensile force at a low tension level for a low tension time period; and
altering at least one of said high tension level, said high tension time period, said low tension level, and said low tension time period in at least one of said cycles.

2. The method of claim 1, wherein said altering step includes altering at least one of said high tension level, said high tension time period, said low tension level, and said low tension time period in a plurality of temporally adjacent cycles.

3. The method of claim 1, wherein said altering step includes increasing or decreasing at least one of said high tension level, said high tension time period, said low tension level, and said low tension time period.

4. The method of claim 1, wherein at least one of said high tension level and said low tension level increase or decrease in each of a plurality of temporally adjacent cycles by a predetermined amount.

5. The method of claim 1, wherein said altering step includes modulating an amplitude of at least one of said high tension level and said low tension level.

6. The method of claim 5, wherein said altering step includes altering a frequency of said amplitude.

7. The method of claim 1, further including applying a corrective tensile force in order to correct for at least one of an unexpected and an expected change in said tensile force.

8. The method of claim 1, wherein said altering step includes altering at least one of said high tension level, said high tension time period, said low tension level, and said low tension time period by applying at least one of a non-linear, curvilinear, sinusoidal, exponential, and logarithmic function to at least one of a previous high tension level, a previous high tension time period, a previous low tension level, and a previous low tension time period.

9. A computer-readable storage medium including a set of instructions for a computer, said instructions including:
a spinal decompression treatment profile generating routine configured to calculate a plurality of cycles of a spinal decompression treatment profile, each of said cycles including an application of a tensile force at a high tension level for a high tension time period and an application of said tensile force at a low tension level for a low tension time period; and
a treatment cycle adjustment routine configured to alter at least one of said high tension level, said high tension time period, said low tension level, and said low tension time period in at least one of said cycles.

10. The set of instructions of claim 9, wherein said treatment cycle analyzing routine is configured to alter at least one of said high tension level, said high tension time period, said low tension level, and said low tension time period in each of a plurality of temporally adjacent cycles.

11. The set of instructions of claim 9, wherein said treatment cycle adjustment routine is configured to increase or decrease at least one of said high tension level, said high tension time period, said low tension level, and said low tension time period.

12. The set of instructions of claim 9, wherein said treatment cycle adjustment routine is configured to increase or decrease at least one of said high tension level and said low tension level in each of a plurality of temporally adjacent cycles by a predetermined amount.

13. The set of instructions of claim 9, wherein said treatment cycle adjustment routine is configured to modulate an amplitude of at least one of said high tension level and said low tension level.

14. The set of instructions of claim 13, wherein said treatment cycle adjustment routine is configured to alter a frequency of said amplitude.

15. The set of instructions of claim 9, further including a tensile force feedback routine configured to measure at least one of an expected tensile force, an unexpected tensile force, and a corrective tensile force, said corrective tensile force capable of being applied during said profile in order to correct for at least one of said expected tensile force and said unexpected tensile force.

16. The set of instructions of claim 9, wherein said treatment cycle adjustment routine is configured to alter at least one of said high tension level, said high tension time period, said low tension level, and said low tension time period by calculating at least one of a non-linear, curvilinear, sinusoidal, exponential, and logarithmic function to be applied to at least one of a previous high tension level, a previous high tension time period, a previous low tension level, and a previous low tension time period.

17. A spinal decompression system for applying a treatment profile in spinal decompression therapy, said system including:
a patient interface device capable of being employed to apply a tensile force to a spine throughout a plurality of cycles in said treatment profile, each of said cycles including an application of said tensile force at a high tension level for a high tension time period and an application of said tensile force at a low tension level for a low tension time period; and
a control system configured to adjust said tensile force applied by said patient interface device by altering at least one of said high tension level, said high tension time period, said low tension level, and said low tension time period in at least one of said cycles.

18. The system of claim 17, wherein said control system is configured to alter at least one of said high tension level, said high tension time period, said low tension level, and said low tension time period in a plurality of temporally adjacent cycles.

19. The system of claim 17, further including a tensile force feedback system capable of calculating a corrective tensile force to be applied to said spine in order to correct for at least one of an unexpected and an expected change in said tensile force

20. The system of claim 18, wherein said control subsystem is configured to adjust said tensile force in each of a plurality of temporally adjacent cycles by one or more of:
calculating at least one of an increase and a decrease in each of said plurality of temporally adjacent cycles by a predetermined amount;
modulating an amplitude of at least one of said high tension level and said low tension level; and
adjusting a frequency of said modulated amplitude.
